(19) United States
(12) Patent Application Publication
(10) Pub. No.: US 2025/0360113 A1
(43) Pub. Date: Nov. 27, 2025

(54) ADMINISTRATION OF DHB TO INCREASE BENEFIT OF LOSING WEIGHT OF INTERMITTENT FASTING

(71) Applicant: NANJING NUTRABUILDING BIO-TECH CO., LTD., Nanjing (CN)

(72) Inventors: Ronghua YI, Nanjing (CN); Shawn WELLS, Nanjing (CN); Kylin LIAO, Nanjing (CN)

(21) Appl. No.: 19/289,986

(22) Filed: Aug. 4, 2025

Related U.S. Application Data
(63) Continuation of application No. PCT/CN2024/075733, filed on Feb. 4, 2024.

(30) Foreign Application Priority Data
Feb. 9, 2023 (WO) PCT/CN2023/075135

(51) Int. Cl. A61K 31/4375; A61P 3/04; A61P 3/06; A61P 3/10; A61P 5/50

(52) U.S. Cl. CPC A61K 31/4375; A61P 3/04; A61P 3/06; A61P 3/10; A61P 5/50

(57) ABSTRACT

The present invention provides methods for increasing a benefit of losing weight of a mammal treated with every-other-day fasting (EODF), comprising administrating to the mammal an effective amount of Dihydroberberine, an analog or derivative thereof, or a pharmaceutically acceptable salt, acid, ester, polymer, analog or derivative thereof.

### CROSS REFERENCE TO RELATED APPLICATION

This application is a continuation application of International Patent Application No. PCT/CN2024/075733, filed on Feb. 4, 2024, which claims the priority of International Patent Application No. PCT/CN2023/075135, filed on Feb. 9, 2023, the contents of all of which are incorporated herein by reference in their entirety.

### FIELD OF THE INVENTION

This invention generally relates to the field of weight loss or control, and more specifically relates to compositions and methods for providing or increasing a benefit of losing weight (e.g., associated with every-other-day fasting) of a mammal in need thereof, with administration of a therapeutically effective amount of DHB, an analog or derivative thereof, or a pharmaceutically acceptable salt, acid, ester, analog or derivative thereof, as an active ingredient.

### BACKGROUND

Metabolic dysfunction (like obese, etc.) is an important risk factor for life threatening diseases that can target cardiovascular, metabolic and hepatic systems. Nowadays, the prevalence of metabolic dysfunction is rapidly increasing in developed and developing countries, leading to increased morbidity and mortality due to type 2 diabetes mellitus, non-alcoholic fatty liver disease, and cardiovascular disease (Abdelaal, M., C. W. le Roux, and N. G. Docherty, Morbidity and mortality associated with obesity. Annals of translational medicine, 2017. 5(7): p. 161.).

Every-other-day intermittent fasting (known as intermittent fasting, EODF), often refers to time-restricted eating, has become the latest diet craze not only for weight loss but also for enhancing health. Everyone from well-known celebrities to everyday, average people are trying EODF and claiming it is successful. Moreover, many literatures have indicated that EODF is able to lower fasted insulin levels, improve glucose tolerance, and lower blood cholesterol, so as to dramatically reduce obesity, insulin resistance, and hepatic steatosis in humans and rodents. Therefore, many emerging evidences suggest that EODF is a natural and simple strategy to prevent and mitigate metabolic dysregulation, especially for obese. However, EODF treatment has been demonstrated to down regulate lipolysis specifically in white adipose tissues (WAT), mediated by a large decrease in the abundance of the catecholamine receptor (Beta-3 adrenergic receptor, ADRB3, which is most abundant in WAT) (Harney, D. J., et al., Proteomics analysis of adipose depots after intermittent fasting reveals visceral fat preservation mechanisms. Cell reports, 2021. 34(9): p. 108804). In other words, these influences are important for preservation of the visceral lipid store during EODF.

Considering the unexpected preservation of the visceral lipid store during EODF, namely lipolysis resistance, which could compromise the effect of losing weight through EODF. Thus, it is understood that, the importance and meaningful of having available products which reduce or even combat the phenomenon of the lipolysis resistance may be appreciated.

The traditional Chinese medicine, called berberine (BBR), a natural plant alkaloid originally isolated from extracts of Berberis aristata and Coptis chinensis (Huanglian), is widely used for anti-diarrhoea, and has been found to have a variety of metabolic benefits in recent years. Additionally, BBR has been demonstrated to promote brown adipocyte differentiation and brown adipose tissues (BAT) thermogenesis by increasing the transcription of PRDM16 driven via AMPK activation and PGC-1α recruitment. Similarly, another study confirmed that BBR could reduce weight gain, promote thermogenic program in BAT, and increase energy expenditure and adaptive thermogenesis in obese db/db mice. What's more, several studies have investigated the role of dietary supplements, such as metformin and BBR, in Beta-3 adrenergic receptor (ADRB3) signaling, which induces the browning of WAT to increase energy expenditure and decrease fat accumulation especially in belly fat in mammals.

Dihydroberberine (DHB), a hydrogenated derivative of BBR, also has these similar benefits and it works better according to our experiment. Furthermore, in vitro, some scientists found that the intestinal absorption rate of DHB was five times higher than BBR. These findings were then tested in practice on mice, and the researchers found that DHB was better absorbed than BBR, including in the mice lacking the necessary intestinal bacteria needed to break down BBR. That is to say, DHB is a far superior ingredient and has more bioavailable than BBR.

Taken together, in this invention, we found that the synergism of DHB administration and EODF could be considered as a promising and attractive ingredient to promote and enhance the benefit of weight loss.

### SUMMARY

This summary is provided to introduce a selection of concepts in a simplified form that is further described below in the Detailed Description. This summary is not intended to identify key features or essential features of the claimed subject matter, nor is it intended to be used to limit the scope of the claimed subject matter.

The present invention generally relates to compositions and methods for providing or increasing a benefit of losing weight of a mammal—particularly a mammal treated with every-other-day fasting (EODF), comprising administrating to the mammal (e.g., engaged in an EODF regimen) an effective amount of Dihydroberberine (DHB), an analog or derivative thereof, or a pharmaceutically acceptable salt, acid, ester, polymer, analog or derivative thereof. The present invention also relates to compositions and methods for losing weight in a mammal, comprising administrating to the mammal an effective amount of Dihydroberberine (DHB), an analog or derivative thereof, or a pharmaceutically acceptable salt, acid, ester, polymer, analog or derivative thereof, while the mammal is engaged in an every-other-day-fasting (EDOF) regimen.

According to this invention, it was surprisingly found that administration of Dihydroberberine (DHB) can particularly increase fat mobilization during EODF in obese mice, thereby increasing the benefit of losing weight of EODF.

One aspect of this invention relates to a method for increasing a benefit of losing weight of a mammal treated with every-other-day fasting (EODF), comprising administrating to the mammal in need thereof an effective amount of Dihydroberberine (DHB), an analog or derivative thereof, or a pharmaceutically acceptable salt, acid, ester, polymer, analog or derivative thereof.

In some embodiments, the benefit comprises reducing a body fat percentage of the mammal, reducing the weight of the mammal, lowering a blood glucose of the mammal, decreasing a blood triglyceride level of the mammal, decreasing a blood total cholesterol level of the mammal, decreasing a blood low-density lipoprotein level of the mammal, decreasing a blood very low-density lipoprotein level of the mammal, improving leptin resistance of the mammal, and/or improving insulin resistance of the mammal.

In some embodiments, the benefit of losing weight of the EODF regimen is greater with administration of Dihydroberberine than the same EODF regimen without administration of the Dihydroberberine.

In some embodiments, the dihydroberberine or its analog or its derivatives are administrated in an amount ranging from 10 mg/day-2000 mg/day. Preferably, it is administered at daily doses of 1 mg to 1000 mg, 5 mg to 800 mg, and 10 mg to 500 mg.

In some embodiments, the daily dose is administered in divided doses or a single dose.

In some embodiments, the administration is at least once a day or more times a day.

In some embodiments, the dihydroberberine is administrated in a form of aqueous solution, aqueous suspension, capsule, drop, granule, liquid, powder, syrup, tablet, functionalized food, beverage, toothpaste, or sublingual articles.

In some embodiments, the dihydroberberine is administrated orally, by intravenous injection, by intramuscular injection, intraperitoneally or sublingually.

In some embodiments, the dihydroberberine is in an ingestible composition. The ingestible composition is selected from the group consisting of a bioceutical composition, a dietary supplement, a medicated feed, a nutraceutical composition, and a pharmaceutical composition.

In some preferred embodiments, the dihydroberberine is prepared in a form of nutritional, drinking, or pharmaceutical composition, for use in a food, drink, nutritional, or pharmaceutical products.

In some embodiments, the mammal suffers from metabolic dysfunctions.

In some embodiments, the mammal is a human or animal (e.g., pet or cattle).

In some embodiments, dihydroberberine (DHB) or its analog or its derivatives are salts, polymers, esters, or acid.

Another aspect of the present invention provides a composition for increasing a benefit of losing weight of a mammal in need thereof, wherein the composition comprises an effective amount of dihydroberberine, an analog or derivative thereof, or a pharmaceutically acceptable salt, acid, ester, analog or derivative thereof.

In some embodiments, the mammal is treated with every-other-day fasting (EODF).

In some embodiments, the benefit comprises reducing a body fat percentage of the mammal, reducing the weight of the mammal, lowering a blood glucose of the mammal, decreasing a blood triglyceride level of the mammal, decreasing a blood total cholesterol level of the mammal, decreasing a blood low-density lipoprotein level of the mammal, decreasing a blood very low-density lipoprotein level of the mammal, improving leptin resistance of the mammal, and/or improving insulin resistance of the mammal.

In some embodiments, the dihydroberberine or its analog or its derivatives are administrated in an amount ranging from 10 mg/day-2000 mg/day. Preferably, it is administered at daily doses of 1 mg to 1000 mg, 5 mg to 800 mg, and 10 mg to 500 mg.

In some embodiments, the daily dose is administered in divided doses or a single dose.

In some embodiments, the administration is at least once a day or more times a day.

In some embodiments, the dihydroberberine is administrated in a form of aqueous solution, aqueous suspension, capsule, drop, granule, liquid, powder, syrup, tablet, functionalized food, beverage, toothpaste, or sublingual articles.

In some embodiments, the dihydroberberine is prepared in a form of nutritional, drinking, or pharmaceutical composition, for use in a food, drink, nutritional, or pharmaceutical products.

Another aspect of the present invention provides a use of dihydroberberine for preparing a composition for increasing a benefit of losing weight of a mammal in need thereof, wherein the composition comprises an effective amount of dihydroberberine, an analog or derivative thereof, or a pharmaceutically acceptable salt, acid, ester, analog or derivative thereof.

In some embodiments, the mammal is treated with every-other-day fasting (EODF).

In some embodiments, the benefit comprises reducing a body fat percentage of the mammal, reducing the weight of the mammal, lowering a blood glucose of the mammal, decreasing a blood triglyceride level of the mammal, decreasing a blood total cholesterol level of the mammal, decreasing a blood low-density lipoprotein level of the mammal, decreasing a blood very low-density lipoprotein level of the mammal, improving leptin resistance of the mammal, and/or improving insulin resistance of the mammal.

In some embodiments, the benefit of losing weight of the EODF regimen is greater with administration of Dihydroberberine than the same EODF regimen without administration of the Dihydroberberine.

### BRIEF DESCRIPTION OF THE DRAWINGS

FIG. 1 is the average body weight of mice in all test groups.

FIG. 2 is the percentage of body fat of mice in all test groups.

FIG. 3 is the weight of subcutaneous fat of mice in all test groups.

FIG. 4 is the weight of para-renal fat of mice in all test groups.

FIG. 5 is the results of TG in all test groups.

FIG. 6 is the results of TCHO in all test groups.

FIG. 7 is the results of LDL in all test groups.

FIG. 8 is the results of VLDL in all test groups.

FIG. 9 is the serum levels of insulin in all test groups.

FIG. 10 is the serum levels of leptin in all test groups.

FIG. 11 is the changes of blood glucose at different time points in oral glucose tolerance test.

FIG. 12 is the area under the curve (AUC) of GTT.

FIG. 13 is the changes of blood glucose at different time points in insulin tolerance test (ITT).

FIG. 14 is the area under the curve (AUC) of ITT.

### DETAILED DESCRIPTION

Reference will now be made in detail to the preferred embodiments of the invention, examples of which are further illustrated. While the invention will be described in conjunction with the preferred embodiments, it will be understood that they are not intended to limit the invention to these embodiments. On the contrary, the invention is intended to cover alternatives, modifications, and equivalents, which may be included within the spirit and scope of the invention as defined by the claims. Furthermore, in the detailed description of the present invention, numerous specific details are set forth in order to provide a thorough understanding of the present invention. However, it will be obvious to one of ordinary skill in the art that the present invention may be practiced without these specific details. In other instances, well known methods, procedures, components, and other features have not been described in detail as not to unnecessarily obscure aspects of the present invention.

As used herein, the term “or” is meant to include both “and” and “or.” In other words, the term “or” may also be replaced with “and/or.”

As used herein, the singular forms “a,” “an” and “the” are intended to include the plural forms as well, unless the context clearly indicates otherwise.

As used herein, the term “comprise” or “include” and their conjugations, refer to a situation where said terms are used in their non-limiting sense to mean that items following the word are included, but items not specifically mentioned are not excluded. It also encompasses the more limiting verb ‘to consist essentially of’ and ‘to consist of’.

As used herein, the term “pharmaceutically acceptable” means pharmaceutically, physiologically, alimentarily, or nutritionally acceptable, and refers to those compositions or combinations of agents, materials, or compositions, and/or their dosage forms, which are within the scope of sound medical judgment, suitable for use in contact with the tissues of human beings and animals without excessive toxicity, irritation, allergic response, or other problem or complication, commensurate with a reasonable benefit/risk ratio.

As used herein, the term “effective amount” refers to an amount that is required to achieve the effect as taught herein. It will be understood, however, that the total daily usage of dihydroberberine (DHB) or its analog or its derivatives may be decided by the attending physician within the scope of sound medical judgment. The specific therapeutically effective dose level for any particular subject will depend upon a variety of factors including the disorder being treated and the severity of the disorder; the specific composition employed, the age, body weight, general health, sex and diet of the subject; the time of administration, route of administration, and rate of excretion of dihydroberberine (DHB) or its analog or its derivatives employed; the duration of the treatment; drugs used in combination or coincidental with dihydroberberine (DHB) or its analog or its derivatives; and like factors well known in the medical arts. For example, it is well known within the skill of the art to start doses of the compound at levels lower than those required to achieve the desired therapeutic effect and to gradually increase the dosage until the desired effect is achieved.

One of skill in the art recognizes that an amount may be considered therapeutically “effective” even if the condition is not totally eradicated or prevented, but it or its symptoms and/or effects are improved or alleviated partially in the subject.

In some embodiments, the composition includes from about 5% to about 10%, 20%, 30%, 40%, 50%, 60%, 70%, 80%, 90%, or 95% by weight of dihydroberberine (DHB) or its analog or its derivatives, and preferably from about 30% to about 90% by weight of dihydroberberine (DHB) or its analog or its derivatives, based upon the total weight of the composition taken as 100% by weight.

As used herein, the term “mammal” or “subject” may be used interchangeably to refer to any animal to which the presently disclosed methods and compositions may be applied or administered. The animal may have an illness or other disease, but the animal does not need to be sick to benefit from the presently disclosed methods and compositions. “Mammal” includes, without limitation, mice, rats, rabbits, guinea pigs, dogs, cats, sheep, goats, cows, horses, primates, such as monkeys, chimpanzees, and apes, and, in particular, humans.

As used herein, “pet” includes, without limitation, mice, rats, rabbits, guinea pigs, dogs, cats, and sheep.

Generally speaking, various embodiments of the present invention provide for providing a mammal with a benefit associated with every-other-day fasting (EODF), or increasing a benefit of losing weight of a mammal treated with every-other-day fasting (EODF), comprising administrating to the mammal (e.g., engaged in an EODF regimen) an effective amount of dihydroberberine, an analog or derivative thereof, or a pharmaceutically acceptable salt, acid, ester, polymer, analog or derivative thereof. It was surprisingly found that administration of dihydroberberine can particularly increase fat mobilization during EODF in obesity mice, thereby increasing the benefit of losing weight of EODF. For instance, the benefit may include reducing a body fat percentage of the mammal, reducing the weight of the mammal, lowering a blood glucose of the mammal, decreasing a blood triglyceride level of the mammal, decreasing a blood total cholesterol level of the mammal, decreasing a blood low-density lipoprotein level of the mammal, decreasing a blood very low-density lipoprotein level of the mammal, improving leptin resistance of the mammal, and/or improving insulin resistance of the mammal.

DHB or its analog or its derivatives are administrated at a daily dose of 10 mg-2000 mg. The dosage may range broadly, depending upon the desired effects and the therapeutic indication. The daily dosage regimen for an adult human patient may be, for example, an oral dose of between 0.01 mg and 3000 mg of dihydroberberine or its metabolites, preferably between 1 mg and 700 mg, e.g., 5 to 200 mg, or between about 0.1 mg and about 1,000 mg of dihydroberberine or its metabolites per kg of body weight of the subject. The dosage may be a single one or a series of two or more given in the course of one or more days, as is needed by the subject. In some embodiments, the compounds are administered for a period of continuous therapy, for example for a week or more, or for months or years. In some embodiments, dihydroberberine or its metabolites, or a pharmaceutically acceptable salt thereof, can be administered less frequently compared to the frequency of administration of an agent within the standard of care. In some embodiments, dihydroberberine or its metabolites, or a pharmaceutically acceptable salt thereof, can be administered one time per day. In some embodiments, the total time of the treatment regime with dihydroberberine or its metabolites, or a pharmaceutically acceptable salt thereof, can be less compared to the total time of the treatment regime with the standard of care.

In some embodiments, the daily dose is administered in divided doses or a single dose. In some embodiments, the administration is at least once a day or more times a day. In some embodiments, the administration is at least 7 days and above in one period.

Multiple techniques of administering a composition exist in the art including, but not limited to, oral, rectal, topical, aerosol, injection and parenteral delivery, including intramuscular, subcutaneous, intravenous, intramedullary injections, intrathecal, direct intraventricular, intraperitoneal, intranasal and intraocular injections. One may also administer the composition in a local rather than systemic manner, for example, via injection of the compound directly into the infected area, often in a depot or sustained release formulation. Furthermore, one may administer the composition in a targeted drug delivery system, for example, in a liposome coated with a tissue specific antibody. The liposomes will be targeted to and taken up selectively by the organ.

The following examples are illustrative of select embodiments of the present invention and are not meant to limit the scope of the invention.

### Examples

50 C57BL/6 male mice aged about 8 weeks, are randomly assigned to five study groups (GA, GB1, GB2, GB3, GB4) of ten mice each (one cage, n=5 per condition). Mice were maintained in a pathogen free condition under a strict 12 h light/dark cycle (0600/1800 h) with 12-15 cycles/hour of air change. Mice were provided with access to respective feed and water ad libitum as per experimental conditions. Mice were housed under controlled laboratory conditions of temperature and humidity at 23±2° C. and 50±10% RH, respectively.

Each study group consisted of ten animals, and treatment details of the individual study groups are given below:
Group A (GA, NC): Normal mice, maintained on normal diet (standard chow, 12% calories from fat; 23% calories from protein and 65% calories from carbohydrates) for 8 weeks, receiving neither EODF nor DHB treatment.
Group B1 (GB1): Obese mice, maintained on high fat diet (HFD, 60% calories from fat, 20% calories from protein, 20% calories from carbohydrate) for 8 weeks, receiving neither EODF nor DHB treatment.
Group B2 (GB2): Obese mice, maintained on HFD for 8 weeks, receiving only EODF and no DHB treatment.
Group B3 (GB3): Obese mice, maintained on HFD for 8 weeks, receiving only DHB treatment and no EODF.
Group B4 (GB4): Obese mice, maintained on HFD for 8 weeks, receiving DHB treatment and EODF combination.

The total experimental time is 19 weeks, including two periods: an elevent-week induction period and a subsequent eight-week treatment period.

At the end of the study, all mice were sacrificed using CO₂ suffocation, all relevant tissues were dissected, weighed, immediately snap frozen in liquid nitrogen, and stored at −80° C.

### EODF Model:

HFD fed male mice in groups at 8 weeks of age were randomly assigned into either ad libitum, or EODF groups on a per cage basis. All cage bedding was changed to paper bedding for the duration of the model. Mice in the EODF group had total deprivation of food and ad libitum access to water from 1200 h-1200 h on alternate days with ad libitum food and water access. EODF cages were changed upon induction of fasting. Ad libitum control mice cages were changed every other day with fresh food provided.

### DHB Administration:

DHB was administered daily via intragastric injection (i.g.) according to a level of 70 mg/kg body weight. Mice not receiving DHB were given orally with ultrapure water.

The synergism of DHB administration and EODF could change the body composition.

Each mouse's body weight is measured weekly during the study at 1200 h. Each animal's body composition was measured and recorded before the treatment and weekly during the study at 1200 h by Nuclear Magnetic Resonance Body Composition Analyzer in accordance with manufacturer's instruction. After sacrifice, subcutaneous fat and para-renal fat were weighed.

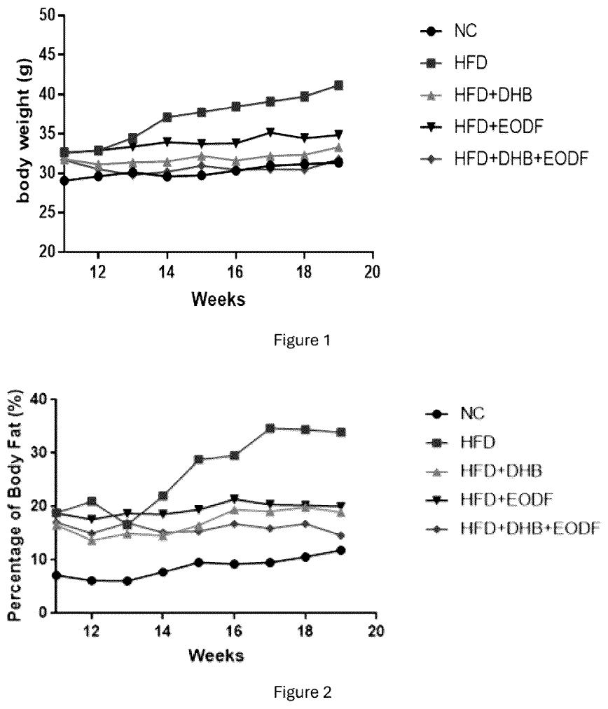

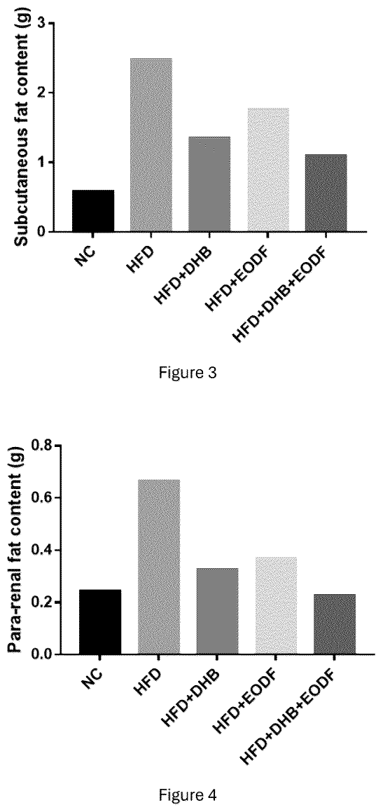

FIG. 1 is the average body weight of mice in all test groups (Body weight were recorded from the beginning of the experiment). FIG. 2 is the percentage of body fat of mice in all test groups (Body weight were recorded from the beginning of the experiment). FIGS. 3-4 are the weight of subcutaneous fat and para-renal fat of mice in all test groups.

As shown in FIGS. 1-4, over the course of the study, HFD fed mice in groups receiving DHB supplementation with or without EODF undergo a greater reduction in body composition, relative to mice subjected to the same HFD diet with and without EODF regimen but not receiving DHB supplementation, indicating that supplementing DHB can achieve or even exceed the beneficial effects of EODF on weight loss. Additionally, the combination of EODF regimen and supplementing DHB can improve the body composition, such as reducing the body weight, body fat, subcutaneous fat and para-renal fat in obese mice than EODF only. This effect persists for as long as the test protocol is continued.

The synergism of DHB administration and EODF could improve biomarkers related to lipid mobilization in mice.

At the end of the study blood was collected from all test groups and a various clinical parameter: plasma total cholesterol (TCHO), triglyceride (TG), low density lipoprotein (LDL), very low-density lipoprotein (VLDL) were measured using corresponding kits according to the manufacturers' instructions.

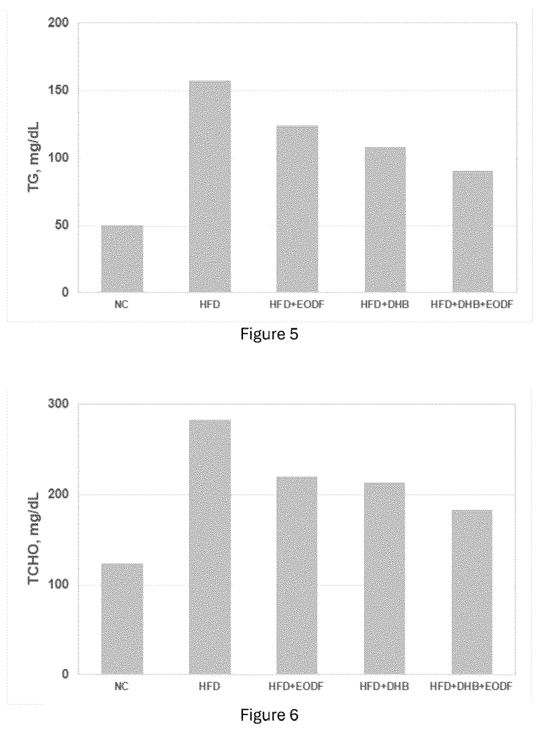

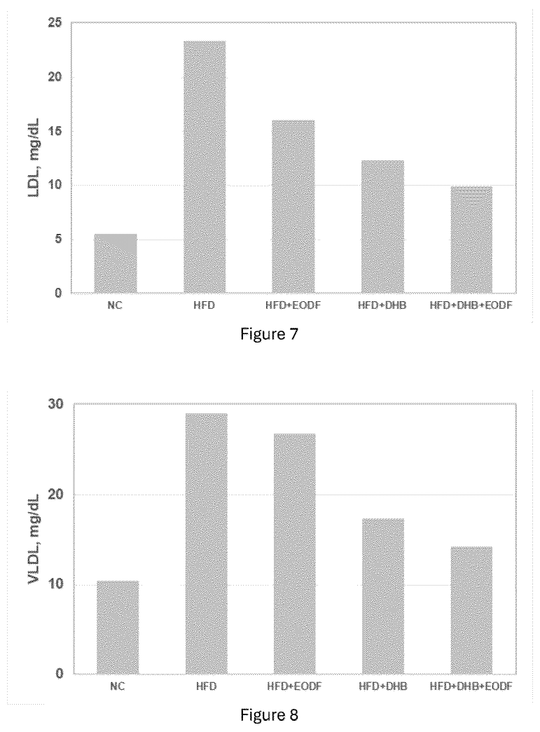

FIGS. 5-8 are the results of TG, TCHO, LDL and VLDL in all test groups, respectively. In FIG. 5-8, it is observed that mice in groups receiving DHB supplementation with or without EODF have lower levels of TG, TCHO, LDL and VLDL relative to mice subjected to the same HFD diet with and without EODF regimen but not receiving DHB supplementation, indicating that supplementing DHB with or without EODF can achieve or even exceed the beneficial effects of EODF on improving biomarkers related to lipid mobilization than EODF only. Additionally, the combination of EODF regimen and supplementing DHB can have greater effects on weight loss than EODF only. This effect persists for as long as the test protocol is continued.

The synergism of DHB administration and EODF could improve leptin resistance and insulin resistance to ameliorate leptin and insulin sensitivity in mice.

Serum levels of insulin and leptin were determined at the end of the treatment period by standard ELISA methods. Since one key feature of obesity is the development of insulin and leptin resistance resulting in an elevation of the circulating levels of these two hormones as a consequence of compensatory physiological responses to insulin and leptin insensitivity, thus leptin is one of the major adipocytokines associated with maintaining glucose homeostasis.

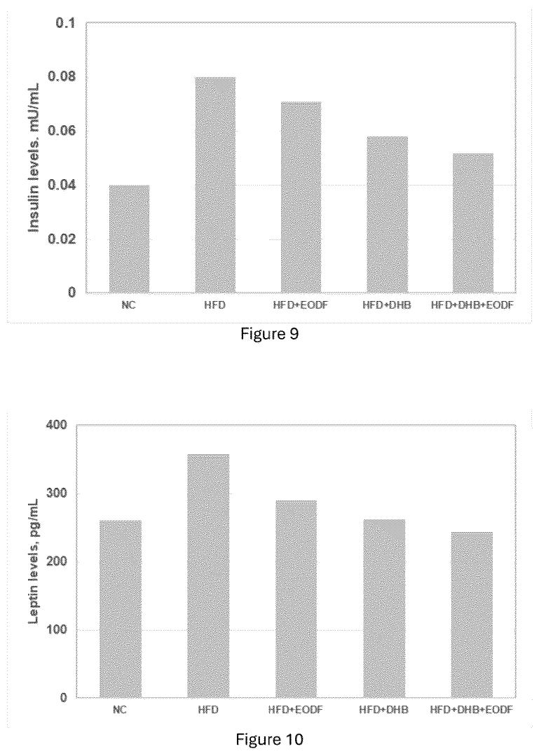

FIGS. 9 and 10 are the serum levels of insulin and leptin in all test groups, respectively. In FIGS. 9 and 10, it is observed that mice in groups receiving DHB supplementation with or without EODF have improved leptin resistance and insulin resistance relative to mice subjected to the same HFD diet with and without EODF regimen but not receiving DHB supplementation, indicating that supplementing DHB can achieve or even exceed the beneficial effects of EODF on improving leptin resistance and insulin resistance of obese mice than EODF only. Additionally, EODF regimen combines with supplementing DHB can have greater effects on improving leptin resistance and insulin resistance of obese mice than EODF only. This effect persists for as long as the test protocol is continued.

The synergism of DHB administration and EODF could reduce blood glucose and insulin in mice.

Oral glucose tolerance test (OGTT): To measure glucose levels in blood, GTT were implemented in the mice (NC and HFD groups) after overnight by using a glucose analyzer. The glucose concentrations were measured in blood collected by venous bleeding from the tail vein immediately at 0, 15, 30, 60 and 120 min after administered glucose at 2 g/kg orally. Towards the end of the final week of the study, GTT was also performed for all mice (NC and HFD groups).

Insulin tolerance test (ITT): To measure insulin levels in blood, ITT were measured in the 6-h fasting mice (NC and HFD groups) by using a glucose analyzer. The glucose concentrations were measured by venous bleeding at 0, 15, 30, 60 and 120 min after an i.p. injection of human insulin at 0.75 U/kg. Towards the end of the final week of the study, ITT was performed for all mice (NC and HFD groups).

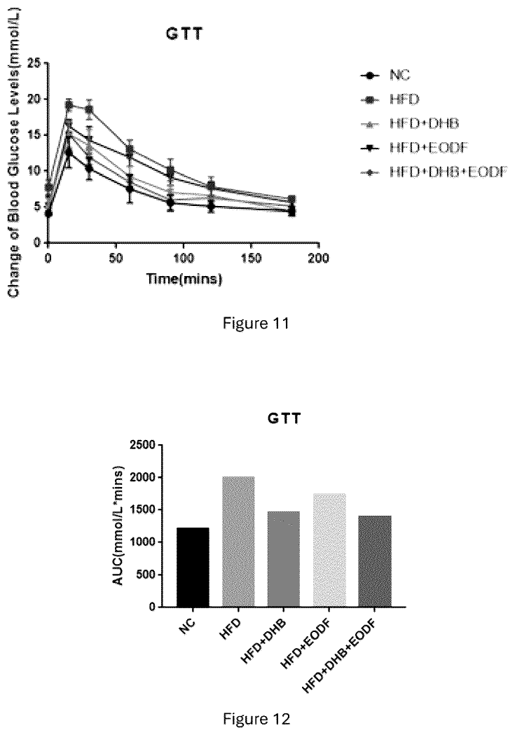

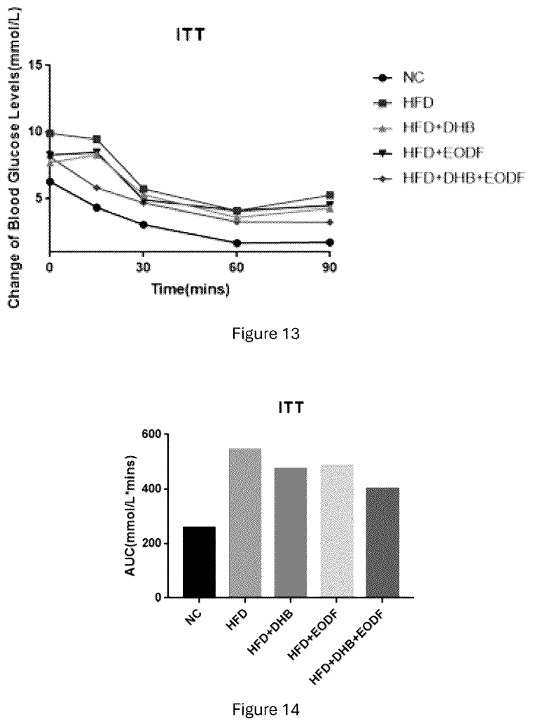

FIGS. 11 and 12 are oral glucose tolerance test (OGTT) observed the changes of blood glucose at different time points and area under the curve (AUC) of GTT. FIGS. 13 and 14 insulin tolerance test (ITT) observed the changes of blood glucose at different time points, and area under the curve (AUC) of ITT.

FIGS. 11-14 demonstrated that, over the course of the study, mice in groups receiving DHB supplementation undergone a greater reduction in blood glucose and insulin levels, maintained the blood glucose and insulin levels of obese mice at a stable level and near to the level of normal mice (GA) throughout the experimental period, and showed better glucose and insulin tolerance relative to mice subjected to the same HFD diet with and without EODF regimen but not receiving DHB supplementation, indicating that supplementing DHB with or without EODF can achieve or even exceed the beneficial effects of EODF on reducing the blood glucose and insulin levels, improving the glucose and insulin tolerance of obese mice than EODF only. Additionally, EODF regimen combines with supplementing DHB can have greater effects on reducing the blood glucose and insulin levels, improving the glucose and insulin tolerance of obese mice than EODF only. This effect persists for as long as the test protocol is continued.

The synergism of DHB administration and EODF could up-regulate lipolysis specifically in WAT, mediated by regulating the abundance of the ADRB3 in mice.

The mRNA levels of ADRB3 protein from inguinal WAT were determined by qRT-PCR.

It is observed that mice in groups receiving DHB supplementation with or without EODF have upregulated lipolysis specifically in WAT by increasing ADRB3 abundance, relative to mice subjected to the same HFD diet with and without EODF regimen but not receiving DHB supplementation, indicating that supplementing DHB can achieve or even exceed the beneficial effects of EODF on improving belly loss of obese mice than EODF only. Additionally, EODF regimen combines with supplementing DHB can have greater effects on triggering lipolysis specifically in WAT of obese mice than EODF only. This effect persists for as long as the test protocol is continued.

40 C57BL/6 male mice aged about 8 weeks, are randomly assigned to four study groups (GA1, GA2, GA3, GA4) of ten mice each (one cage, n=4 per condition). Mice were maintained in a pathogen free condition under a strict 12 h light/dark cycle (0600/1800 h) with 12-15 cycles/hour of air change. Mice were provided with access to respective feed and water ad libitum as per experimental conditions. Mice were housed under controlled laboratory conditions of temperature and humidity at 23±2° C. and 50±10% RH, respectively.

Each study group consisted of ten animals, and treatment details of the individual study groups are given below:
Group A1 (GA1, NC): Normal mice, maintained on normal diet (standard chow, 12% calories from fat; 23% calories from protein and 65% calories from carbohydrates) for 8 weeks, receiving neither EODF nor DHB treatment.
Group A2 (GA2): Normal mice, maintained on normal diet for 8 weeks, receiving only EODF and no DHB treatment.
Group A3 (GA3): Normal mice, maintained on normal diet for 8 weeks, receiving only DHB treatment and no EODF.
Group A4(GA4): Normal mice, maintained on normal diet for 8 weeks, receiving DHB treatment and EODF combination.

Test according to the above method. The results show that over the course of the study, mice in groups receiving DHB supplementation had a greater effectiveness relative to mice subjected to the same normal diet with and without EODF regimen but not receiving DHB supplementation, indicating that supplementing DHB with or without EODF can achieve or even exceed the beneficial effects of EODF. EODF regimen combines with supplementing DHB can have greater effects than EODF only. This effect persists for as long as the test protocol is continued.

Although specific embodiments and examples of this invention have been illustrated herein, it will be appreciated by those skilled in the art that any modifications and variations can be made without departing from the spirit of the invention. The examples and illustrations above are not intended to limit the scope of this invention. Any combination of embodiments of this invention, along with any obvious their extension or analogs, are within the scope of this invention. Further, it is intended that this invention encompass any arrangement, which is calculated to achieve that same purpose, and all such variations and modifications as fall within the scope of the appended claims.

All the features disclosed in this specification (including any accompanying claims, abstract and drawings) may be replaced by alternative features serving the same, equivalent or similar purpose, unless expressly stated otherwise. Thus, unless expressly stated otherwise, each feature disclosed is one example of a generic series of equivalent or similar features.

It is to be understood that while the invention has been described in conjunction with the detailed description thereof and accompanying figures, the foregoing description and accompanying figures are only intended to illustrate, and not limit the scope of the invention, which is defined by the scope of the appended claims. Other aspects, advantages, and modifications are within the scope of the following claims. All publications referenced herein are incorporated by reference in their entireties.

What is claimed is:

1. A method for increasing a benefit of losing weight of a mammal treated with every-other-day fasting (EODF), comprising administrating to the mammal in need thereof an effective amount of Dihydroberberine (DHB), an analog or derivative thereof, or a pharmaceutically acceptable salt, acid, ester, polymer, analog or derivative thereof.

2. The method of claim 1, wherein the benefit comprises reducing a body fat percentage of the mammal, reducing the weight of the mammal, lowering a blood glucose of the mammal, decreasing a blood triglyceride level of the mammal, decreasing a blood total cholesterol level of the mammal, decreasing a blood low-density lipoprotein level of the mammal, decreasing a blood very low-density lipoprotein level of the mammal, improving leptin resistance of the mammal, and/or improving insulin resistance of the mammal.

3. The method of claim 1, wherein the benefit of losing weight of the EODF regimen is greater with administration of Dihydroberberine than the same EODF regimen without administration of the Dihydroberberine.

4. The method of claim 1, wherein the dihydroberberine is administrated in an amount ranging from 10 mg/day-2000 mg/day.

5. The method of claim 1, wherein the dihydroberberine is administrated in a form of aqueous solution, aqueous suspension, capsule, drop, granule, liquid, powder, syrup, tablet, functionalized food, beverage, toothpaste, or sublingual articles.

6. The method of claim 1, wherein the dihydroberberine is administrated orally, by intravenous injection, by intramuscular injection, intraperitoneally or sublingually.

7. The method of claim 6, wherein the dihydroberberine is in an ingestible composition.

8. The method of claim 7, wherein the ingestible composition is selected from the group consisting of a bioceutical composition, a dietary supplement, a medicated feed, a nutraceutical composition, and a pharmaceutical composition.

9. The method of claim 1, wherein the dihydroberberine is prepared in a form of nutritional, drinking, or pharmaceutical composition, for use in a food, drink, nutritional, or pharmaceutical products.

10. The method of claim 1, wherein the mammal suffers from metabolic dysfunctions.

11. The method of claim 1, wherein the mammal is engaged in every-other-day fasting (EODF).
